(19) United States
(12) Patent Application Publication
(10) Pub. No.: US 2010/0218766 A1
(43) Pub. Date: Sep. 2, 2010

(54) CUSTOMIZABLE MANDATORY/SPONTANEOUS CLOSED LOOP MODE SELECTION

(75) Inventor: Gary Scott Milne, Louisville, CO (US)

Correspondence Address: NELLCOR PURITAN BENNETT LLC, 6135 GUNBARREL AVENUE, BOULDER, CO, 80301, US

(73) Assignee: Nellcor Puritan Bennett LLC, Boulder, CO (US)

(21) Appl. No.: 12/395,321

(22) Filed: Feb. 27, 2009

(51) Int. Cl. A61M 16/00; G06F 3/00

(52) U.S. Cl. 128/204.23; 715/700

(57) ABSTRACT

A multimode ventilator is disclosed that provides physicians and clinicians the ability to select multiple modes of operation for the ventilator. Embodiments of the present disclosure provide users with the ability to select from multiple modes of ventilator operations. The users may also set transition conditions and monitoring thresholds that determine when the multimode ventilator transitions between the selected modes of operations. Multiple user interfaces are also disclosed that allow the user to interact with the multimode ventilator in order to select particular modes of operation, transition conditions, and monitoring thresholds.

### INTRODUCTION

Medical ventilators are used to aid patients with breathing. Depending on the patient, some mechanical ventilators are configured by the user to provide the entirety of each breath to the patient, or to provide some level of support to a patient's own effort to breathe.

Generally, the modes of ventilation medical ventilators provide can be grouped into two categories: mandatory modes and spontaneous modes. In a mandatory ventilation mode, the medical ventilator completely performs the task of breathing for a patient. Various forms of mandatory mode ventilation exist. In a spontaneous ventilation mode, the patient is performing at least some breathing on their own; however, the patient may be incapable of providing enough ventilation on his/her own and the medical ventilator is necessary to assist the patient in taking a breath. As with mandatory modes of ventilation, various types of spontaneous mode ventilation are employed to aid patients in breathing.

For some patients, as their condition improves they may become more capable of taking spontaneous breaths. It thus may be desirable to switch medical ventilators between a mandatory mode and a spontaneous mode of operation.

### SUMMARY

The present disclosure describes systems, methods and user interfaces for a multimode ventilator that allows customizable operation by a user. In embodiments, a user is able to specify two different operating modes for a multimode ventilator. The user is also able to specify transition conditions that determine when the ventilator should transition between the modes of operation. The multimode ventilator initiates ventilation in the first mode specified by the user. Upon reaching a transition condition, the ventilator transitions into the other selected mode of operation. In embodiments, a user interface is provided that allows the user to interact with the multimode ventilator. In some embodiments, the user may select between a plurality of mandatory modes of ventilation and a plurality of spontaneous modes of ventilation. In this manner, the clinician or other user can select the preferred modes of ventilation for a particular patient, and are not limited to a single mandatory mode paired with a single spontaneous mode option.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

This summary is provided to introduce a selection of concepts in a simplified form that are further described below in the Detailed Description. This summary is not intended to identify key features or essential features of the claimed subject matter, nor is it intended to be used to limit the scope of the claimed subject matter. It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto.

FIG. 1 is a diagram illustrating an embodiment of a ventilator system utilizing an endotracheal tube for air delivery to the patient's lungs.

FIG. 2 is a is a flow chart representing an embodiment of a method for receiving a plurality of first modes of operation and second modes of operation from a user.

FIG. 3 is an embodiment of a graphical user interface that provides a user the ability to select from a plurality of first and second modes of ventilation operation.

FIG. 4 is an embodiment of a graphical user interface that provides a user the ability to select a control mode of operation and a spontaneous mode of operation for a ventilator.

FIG. 5 is a flow chart representing an embodiment of a method for operating a multimode ventilator with user selected modes of operation.

FIG. 6 illustrates a functional block diagram of modules and other components that may be used in an embodiment of a multimode ventilator.

### DETAILED DESCRIPTION

Before the multimode ventilator methods, systems, and user interfaces are disclosed and described, it is to be understood that this disclosure is not limited to the particular structures, or process steps disclosed herein, but is extended to equivalents thereof as would be recognized by those ordinarily skilled in the relevant arts. It should also be understood that terminology employed herein is used for the purpose of describing particular embodiments only and is not intended to be limiting. It must be noted that, as used in this specification, the singular forms “a,” “an,” and “the” include plural referents unless the context clearly dictates otherwise. Thus, for example, reference to “a step” may include multiple steps. Likewise, reference to “an operation” or “a step” may include multiple operations or steps, respectively.

This disclosure will now more fully describe exemplary embodiments with reference to the accompanying drawings, in which some of the possible embodiments are shown. Other aspects, however, may be embodied in many different forms and the inclusion of specific embodiments in the disclosure should not be construed as limiting such aspects to the embodiments set forth herein. Rather, the embodiments depicted in the drawings are included to provide a disclosure that is thorough and complete and which conveys the intended scope to those skilled in the art. When referring to the figures, like structures and elements shown throughout are indicated with like reference numerals.

Although the techniques introduced above and discussed in detail below may be implemented for a variety of medical devices, the present disclosure will discuss the implementation of these techniques in the context of a medical ventilator for use in providing ventilation support to a human patient. The reader will understand that the terms “medical ventilator” and “ventilator” refer to such devices and are used interchangeably throughout the present disclosure. Additionally, one of skill in the art will understand that the technology described in the context of a medical ventilator for human patients could be adapted for use with other systems such as ventilators for non-human patients and general gas transport systems.

A ventilator is a device that mechanically helps patients breathe by replacing some or all of the muscular effort required to inflate and deflate the lungs. Ventilatory assistance is indicated for certain diseases affecting the musculature required for breathing, such as but not limited to muscular dystrophies, polio, amyotrophic lateral sclerosis (“ALS”), and Guillain-Barré syndrome. Mechanical ventilation may also be required during the sedation associated with surgery and as the result of various injuries, such but not limited to as high spinal cord injuries and head trauma.

Ventilators may provide assistance according to a variety of methods based on the needs of the patient. These methods include volume based and pressure based methods. More specifically, volume based methods may include Volume Control (“VC”), Assist Control (“AC”), Synchronized Intermittent Mandatory Ventilation (“SIMV”), Controlled Mechanical Ventilation (“CMV”), Pressure-Regulated Volume Control (“PRVC”), Auto-Flow techniques, or any other type of volume based ventilation known in the art. Pressure based methods may involve Assist Control (“AC”), Synchronized Intermittent Mandatory Ventilation (“SIMV”), Controlled Mechanical Ventilation (“CMV”), Pressure Support Ventilation (“PSV”), Continuous Positive Airway Pressure (“CPAP”), and Positive End Expiratory Pressure (“PEEP”) techniques, or any other type of pressure based ventilation known to the art. In addition to volume based and pressure based approaches, ventilators may also provide dual mode approaches such as SIMV, AC, VC+, or any other type of dual mode support known to the art.

Ventilation may be achieved by invasive or non-invasive means. Invasive ventilation utilizes an endotracheal tube (“ET tube”) or a tracheostomy tube inserted into the patient's trachea in order to deliver air to the lungs. Non-invasive ventilation may utilize a mask or other device placed over the patient's nose and mouth,

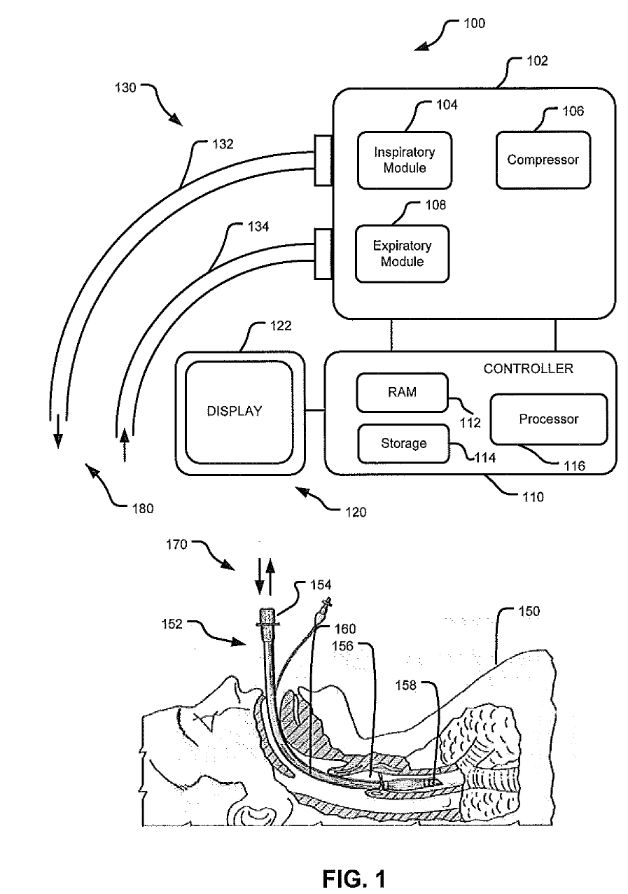

FIG. 1 illustrates an embodiment of a ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via a patient interface 154, illustrated as an endotracheal tube (“ET tube”) 152 although a face mask or other interface may also be used. Air flow is continuous between ventilation tubing system 130 and ET tube 152 and is represented by flow arrows 170 and 180. Ventilation tubing system 130 may be a two-limb or a one-limb (not shown) circuit for carrying gas to and from the patient 150. In a two-limb embodiment as shown, a fitting (not shown), often referred to as a “wye-fitting”, may be provided to couple the patient interface 154 to the inspiratory limb 132 and the expiratory limb 134 of the ventilation tubing system 130.

Pneumatic system 102 may be configured in a variety of ways. In the present embodiment, system 102 includes an expiratory module 108 coupled with an expiratory limb 134 and an inspiratory module 104 coupled with an inspiratory limb 132. Compressor 106 or another source(s) of pressurized gas (e.g., air and oxygen) is coupled with inspiratory module 104 to provide a gas source for ventilatory support via inspiratory limb 132.

The pneumatic system may include a variety of other components, including, but not limited to, sources for pressurized air and/or oxygen, mixing modules, valves, sensors, tubing, accumulators, filters, etc. Controller 110 is operatively coupled with a pneumatic system 102, a signal measurement, an acquisition systems (not shown), and an operator interface 120 may be provided to enable an operator to interact with the ventilator 100 (e.g., change ventilator settings, select operational modes, view monitored parameters, etc.). Controller 110 may include memory 112, one or more processors 116, storage 114, and/or other components of the type commonly found in command and control computing devices.

The memory 112 is computer-readable storage media that stores software that is executed by the processor 116 and which controls the operation of the ventilator 100. In an embodiment, the memory 112 comprises one or more solid-state storage devices such as, for example, flash memory chips. In an alternative embodiment, the memory 112 may be mass storage device connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. Computer-readable storage media includes volatile and non-volatile, removable and non-removable media implemented in any method or technology for storage of information such as computer-readable instructions, data structures, program modules or other data. Computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer. The memory 112 stores the computer executable instructions and/or modules to perform the methods and generate the embodiments of user interfaces disclosed herein. Memory 112 may also be operable to store trending and tracking data for the operation of the ventilator including, but not limited to, the number of times the ventilator has transition between modes of operation.

As described in more detail below, controller 110 issues commands to pneumatic system 102 in order to control the breathing assistance provided to the patient by the ventilator. The specific commands may be based on inputs received from patient 150, pneumatic system 102 and sensors, operator interface 120 and/or other components of the ventilator. In embodiments, controller 110 and operator interface 120 communicate via input/output connections (not shown), Input/output connections are well known in the art and need not be discussed at length here. In embodiments, operator interface includes a display 122 that may be touch-sensitive, enabling the display to serve both as an input and output device. In other embodiments, operator interface 120 is a collection of input and output devices. For example, input devices are user interface selection devices that may include, but are not limited to, a keyboard, a mouse, a pen, a voice input device, a touch input device, etc. Output devices, for example, may be a display such as display 122 which includes, but is not limited to, cathode ray tube displays, plasma screen displays, liquid crystal screen displays, speakers, printers, etc. These input and output devices, either individually or in combination, are connected to controller 110 through input and output connections and are used to collect from and/or display information to a user. For example, operator interface may be used to display the various embodiments of graphical user interfaces (“GUI”) described herein to allow the operator to set the multimode operation of a ventilator. All these described input and output devices are well known in the art and need not be discussed at length.

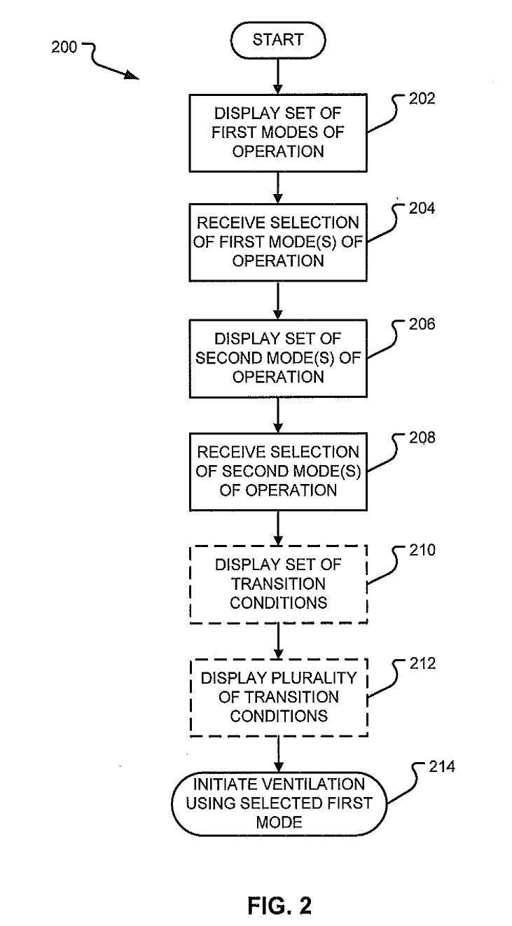

FIG. 2 is a flow chart representing an embodiment of a method 200 for receiving a plurality of first modes of operation and second modes of operation from a user. In embodiments, a ventilator, such as ventilator 100 may perform method 200. In such embodiments, controller 110 may execute the logic necessary to perform method 200 using processor 116. The method 200 begins at operation 202 where a set of first modes of ventilator operations is displayed to a user, such as a physician or a clinician. In embodiments, the first modes of ventilator operations are presented to the user via a display device, such as display 122.

In an embodiment, the first modes of ventilator operation may include any known mode of ventilator operation. For example, the first modes of ventilator operation may be a type of mandatory mode of ventilator operation. Mandatory modes of ventilator operation are used to assist a patient who is unable to breathe under his or her own volition. Examples of mandatory modes, also sometimes referred to as “control modes”, of ventilator operation include, but are not limited to, Volume Controlled Ventilation, Pressure Controlled Ventilation, Air way Pressure Release Ventilation (“APRV”), Biphasic Positive Airway Pressure (“BIPAP) Ventilation, BiLevel Ventilation, and Adaptive Support Ventilation (“ASV”). Volume Controlled Ventilation is a mandatory mode of ventilation where a specific volume of air is delivered to a patient in each breath. Pressure Controlled Ventilation is a mandatory mode of ventilator operation where a specific pressure for delivery is established for the patient. Pressure Control-Inverse Ratio Ventilation (“RC-IRV”) is an example of a specific type of Pressure Controlled Ventilation. APRV Ventilation is a set level of Continuous Positive Airway Pressure (“CPAP”) that intermittently releases to a lower level on a time-controlled basis. BIPAP Ventilation is pressure controlled ventilation that allows unrestricted spontaneous breathing. BiLevel Ventilation is a combination of APRV and BIPAP Ventilation. ASV is a type of closed-loop ventilation. While specific examples of mandatory modes of ventilator operation are provided in this disclosure, one of skill in the art will appreciate that any mandatory modes of ventilator operation, now known to the art or later developed, may be practiced with embodiments of the present disclosure.

The first mode of ventilator operations may also include spontaneous modes of ventilator operation. Spontaneous modes of ventilator operation are used when a patient is able to perform limited breathing and/or the patient is taking spontaneous breaths. Examples spontaneous modes of ventilator operation include, but are not limited to, Pressure Support, Volume Support and Proportional Assist Ventilation (“PAV”), BiLevel Ventilation, which may be practiced as both a mandatory mode and a spontaneous mode. While specific examples of spontaneous modes of ventilator operation are provided in this disclosure, one of skill in the art will appreciate that any spontaneous modes of ventilator operation, now known to the art or later developed, may be practiced with embodiments of the present disclosure.

Flow proceeds to operation 204, where the ventilator receives a selection of a first mode of ventilator operation. For example, a user operating the ventilator inputs a selection of one of the displayed first modes of ventilator operation using operator interface 120. The user may input the selection using a touch screen, a keyboard, a mouse, or any other input device that is a part of operator interface 120. The selected information is relayed to the controller 110 via the input/output connections described with respect to FIG. 1.

At operation 206, the ventilator displays a set of second modes of operation via a display such as display 122. In embodiments, the second set of ventilator operations may include the same operations as the first set of ventilator operations. In an alternate embodiment, the second set of ventilator operations is a subset of the first set of ventilator operations. This is because two modes of ventilator operations may be incompatible with each other. For instance, in certain circumstances it may not be desirable to alternate between two specific modes of operations, such as between two control modes. In such circumstances, if the user selected one of the control modes as the first mode of operation, the second set of ventilator operations may be a subset of the first set of ventilator operations that only includes spontaneous modes of operation or modes of operation compatible with the selected first mode of operation. In an embodiment, the ventilator may automatically determine which modes are compatible with the users selection of the first mode of ventilator operation and only display second modes of operation that are compatible with the first selected mode.

In yet another embodiment, the ventilator may display alternate options to the user at operation 206. For example, the ventilator may present the user the option to turn the ability to transition between modes of ventilator operation off. In other embodiments, the ventilator may display an option to automatically determine a second mode of operation. For example, the automatic determination may be based upon the first mode of ventilator operation, the patient's status, or other factors known in the art. If the user selects this option, the ventilator may automatically select new modes of operation during ventilation. The new selection may be based upon the automatic monitoring of the patient performed by the ventilator.

Flow proceeds to operation 208, where the ventilator receives a selection of a second mode of ventilator operation. As described, a user operating the ventilator inputs a selection of one of the displayed second modes of ventilator operation using operator interface 120. The user may input the selection using a touch screen, a keyboard, a mouse, or any other input device that is a part of operator interface 120.

Upon selecting the first and second modes of operations, transition conditions may be set to determine when to switch between the first and second mode. The transition conditions may be used to determine when the ventilator should transition from the first mode of operation to the second mode of operation. Transition conditions may also be used to determine when to transition back from the second mode of operation to the first mode of operation. In embodiments, transition conditions may be based upon measurements related to airflow, Fraction of Inspired Oxygen (“FIO₂”) percentages, the patient's respiration rate (e.g., establishing a respiratory rate criteria), saturation levels of the air exiting the patients lungs, end-tidal CO₂ or CO levels, a threshold of minimum spontaneous efforts made by the patient (based of pressure and flow readings), Minute Ventilation (“MV”), pressure levels, e.g., P100 levels (pressure generated in the first 1/10^{th} of a second), Saturation of Peripheral Oxygen (“S_{P}O₂”) levels, or any other measurements or calculations known to the art.

In embodiments, the transition conditions and the thresholds for the various transition conditions may be automatically determined by the ventilation system. For example, if the user selects FIO₂ as a transition condition between a control mode of operation and a spontaneous mode of operation, the ventilator may automatically set a threshold FIO₂ percentage to determine when to make the transition. In such circumstances, a transition from a control mode of operation to a spontaneous mode of operation can be made when FIO₂ levels are not too high. For example, the FIO₂ threshold may be set at 40%, such that the ventilator will transition from a control mode to a spontaneous mode when the FIO₂ percentage falls below the 40% threshold.

Continuing the example of transitioning from a mandatory mode to a spontaneous mode, other transition levels may be preset and/or determined by the ventilator. For example, if the selected condition is airflow the ventilator may switch from a control mode to a spontaneous mode if the volume of airflow (Minute Ventilation) is not too high. For example, if airflow is less than 10 liters per minute the ventilator may transition from the control mode to the spontaneous mode. Other instances of switching from a control mode to a spontaneous mode may occur when end-tidal CO2 levels are low, the patient's respiratory rate is low, the S_{P}O₂ level is low, or when the patient's respiratory effort is within an acceptable range. The converse of these examples may be applied when determining when to switch from a spontaneous mode to a control mode. While embodiments of transitioning between first and second modes of operation have been described using specific first and second modes and specific transition conditions, these descriptions are provided for the purpose of describing specific embodiments of the present disclosure and are not intended to limit the scope of the disclosure. One of skill in the art will appreciate that other transition conditions and thresholds may be employed within the scope of the present disclosure.

In other embodiments, the transition conditions may not automatically be determined by the ventilation system. In such embodiments, transition conditions and interface elements (such as text boxes, dropdown menus, radio buttons, etc.) for the input of respective, user-selected threshold levels may be displayed to the user at operation 210. In an embodiment, only certain transition conditions may be applicable to the modes of operation selected by the user in steps 204 and 206 and the ventilator system may only display or allow the user to edit the transition conditions applicable to the selected modes of operation at step 210. In yet another embodiment, transition condition thresholds may be displayed to the user in addition to the transition conditions. In such embodiments, the thresholds may be displayed concurrently with the transition conditions or may be displayed upon the selection of one or more transition conditions.

In further embodiments, a monitoring threshold may be displayed at operation 210. In an embodiment, a transition between a first mode of operation and a second mode of operation may not occur upon first meeting a transition condition. Instead, the transition condition must be consistently maintained for some time interval (which may itself be one of the transition conditions set by the user) before the transition occurs. In such embodiments, a monitoring threshold may be present to ensure that the achieved transition condition is not an error. For example, a time period may be set as a monitoring threshold in which the transition condition is consistently met before switching between modes. Continuing from previous examples, if a patients FIO₂ percentage falls below a 40% threshold as specified by the example transition condition, the FIO₂ percentage must remain below the 40% threshold for a predetermined time (e.g., two minutes) or for a predetermined number of monitored results (e.g., 10 results) before the ventilator transitions between modes. In embodiments, the monitoring threshold may be determined automatically by the ventilator based upon the modes of operation selected, the transition conditions selected, the patient's status, etc. In other embodiments, a monitoring threshold may be displayed at operation 210 thus providing the user the ability to select and/or input a specific monitoring threshold.

In other embodiments, the user may select multiple transition conditions. A first transition condition may be set to specify when the ventilator should transition from the first mode of operation to the second mode of operation. The user may also select a second mode of operation to specify when the ventilator should transition back from the second mode to the first mode. In further embodiments, the user can specify multiple transition conditions specifying when to transition from a first mode of operation to a second mode of operation and vice versa.

Flow then proceeds to step 212 where the ventilator receives a selection of one or more transition conditions, transition condition thresholds, and/or monitoring thresholds from the user. As described, a user operating the ventilator inputs a selection of one of the displayed first modes of ventilator operation using operator interface 120. The user may input the selection using a touch screen, a keyboard, a mouse, or any other input device that is a part of operator interface 120. In embodiments, specific transition conditions may be associated with specific modes of operations. For example, the user may associate a specific transition condition that specifies when the ventilator should transition from the first mode of operation to the second mode of operation and a different transition condition to specify when the ventilator should transition from the second mode of operation back to the first mode of operations. Similarly, in embodiments, different monitoring thresholds may be associated with different transition thresholds.

Upon receiving a selections of the first mode of operation, the second mode of operation, and one or mode transition conditions and thresholds from the user, flow proceeds to operation 214 where ventilation is initiated using the first selected mode of operation. In embodiments, it is not required that the user select all of these described settings before the ventilator operation is initiated. As described, the ventilator may automatically determine certain modes and/or thresholds. Additionally, in other embodiments these modes, transition conditions, and monitoring thresholds may be selected and/or changed by the user during ventilator operation. While the embodiment of the method 200 has been described as discreet steps occurring in a certain order, the description was provided for illustrative purposes only. One of skill in the art will appreciate that these operations may occur in any order. For example, one or more transition conditions may be selected before the first or second modes of operations are selected.

The method 200 provides a user with the ability to set the configuration and operation of the ventilator to best suit the patient's individual needs. By allowing the user to customize modes of operations, transition conditions, and monitoring thresholds, the user is able to set a ventilator to operate in the optimal interests of a patient based upon the patient's particular needs and medical history.

More complicated variations of the method 200 are also considered within the scope of this technology. For example, in yet another embodiment the user may be allowed to selected multiple second modes in the second mode selection operation 208 and further define additional conditions associated with the second modes in the condition selection operation 212 that indicate which of the two second modes the ventilator should transition based on the patient's respiratory conditions at the time. For example, two different spontaneous modes may be selected as second modes and the user may further indicate that one mode is to be used if the patient's work of breathing is weak and the other mode is to be used if the patient's work of breathing is sufficiently strong.

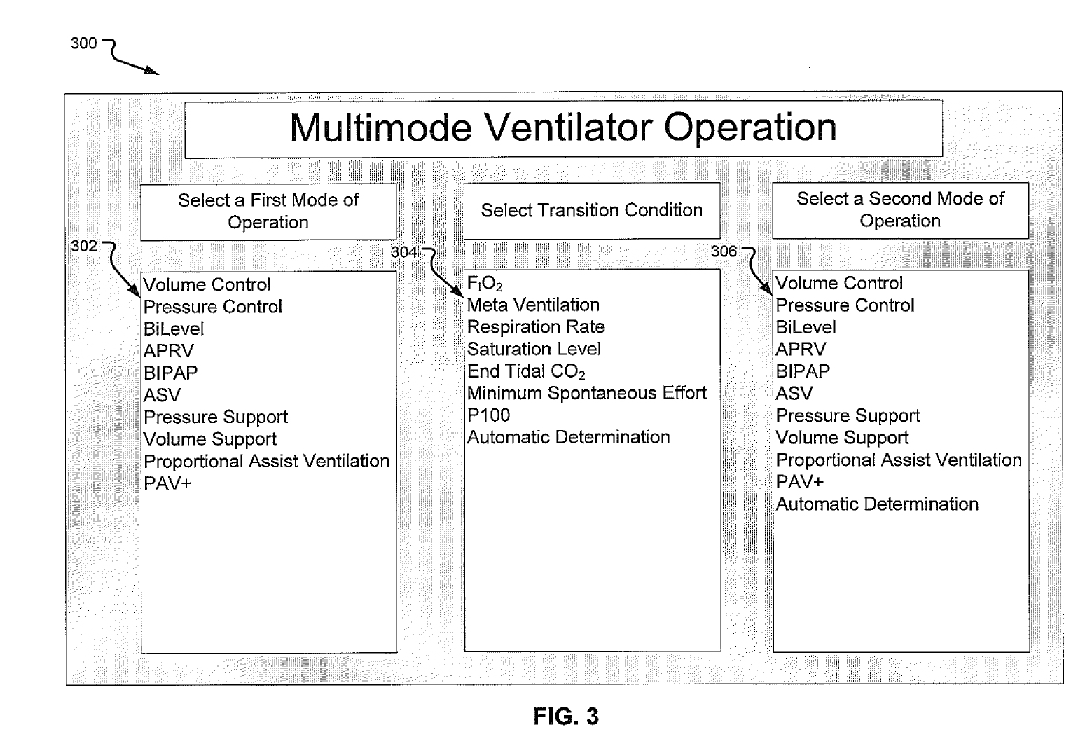

Referring now to FIG. 3, FIG. 3 illustrates an embodiment of a graphical user interface 300 that provides a user the ability to select from a plurality of first and second modes of ventilation operation. In embodiments, the user interface may be generated by the ventilator system using controller 110. The user interface may be displayed on a display such as display 122. User interface 300 includes three separate display areas. Display area 302 displays a set of first modes of ventilator operations according to the embodiments previously described with respect to FIG. 2. A user can select one of the modes of operation from display area 302 using an input device as described in FIG. 1. Display area 304 displays a set of transition conditions as described with respect to the embodiments of FIG. 2. A user can select one or more of the transition conditions from display area 304 using an input device as described in FIG. 1. In other embodiments, an option to let the ventilator automatically determine one or more transition conditions may also be displayed in display area 306. Automatic determination of the one or more transition conditions may be performed as described with respect to FIG. 2. Display area 306 displays a set of second modes of ventilator operations according to the embodiments previously described with respect to FIG. 2. In embodiments, the second set of modes of operation may be the same as the first set of modes of operation or a subset of the first set of operations. In other embodiments, an option to let the ventilator automatically determine a second mode of operation may also be displayed in display area 306. Automatic determination of the second mode of operation may be performed as described with respect to FIG. 2. A user can select one of the modes of operation from display area 302 using an input device as described in FIG. 1,

In other embodiments, transition conditions and their associated values and/or monitoring thresholds may also be displayed on the user interface. One of skill in the art will appreciate that the user interface 300 of FIG. 3 is one embodiment of a user interface that is contemplated by the present disclosure. Other embodiments of user interfaces incorporating the teaching of the present disclosure may practiced. Additionally, while specific examples of first modes of operation, second modes of operation, and transition conditions are provided in display areas 302, 304, and 306, one of skill in the art will appreciate that other modes of operation and other transition conditions are contemplated may be practiced with the teachings of the present disclosure.

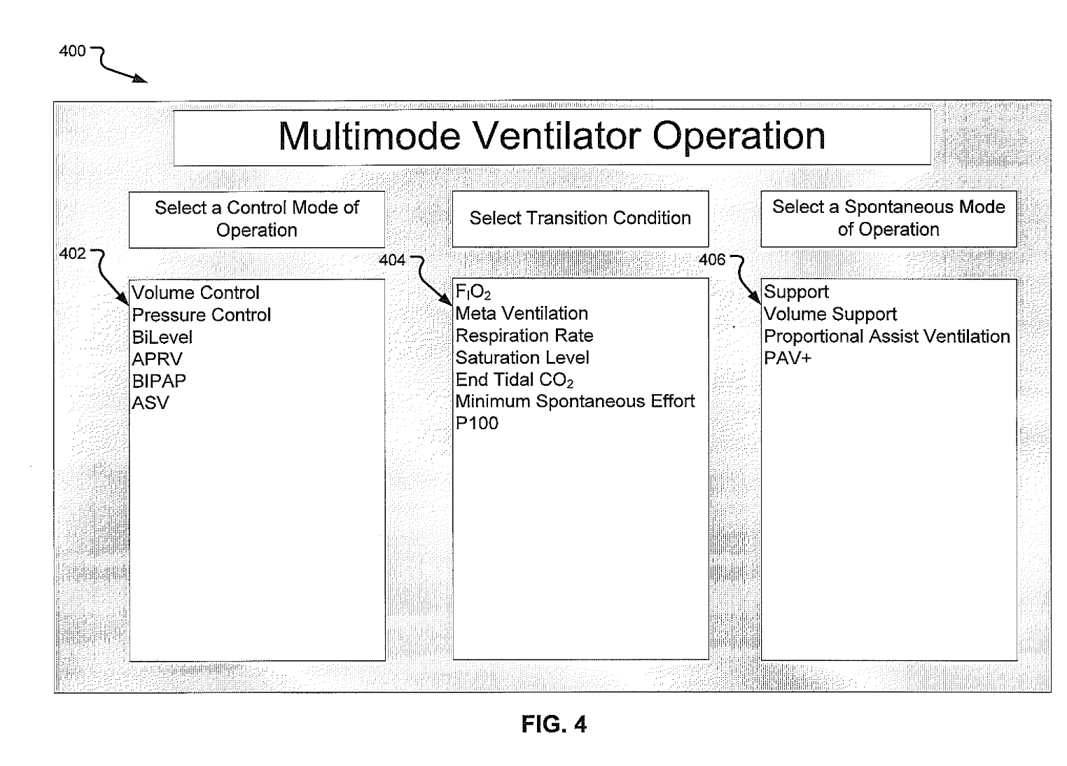

FIG. 4 is an embodiment of a graphical user interface 400 that provides a user the ability to select a mandatory mode of operation and a spontaneous mode of operation for a ventilator. Display area 402 provides a set of control modes of operations that may be selected by a user. Display area 404 provides a set of transition conditions that may be selected by a user. Display area 406 provides a set of spontaneous modes that may be selected by the user. A user can select one or more options from display areas 402, 404, and 406 using an input device as described in FIG. 1.

In other embodiments, transition condition and their associated values and/or monitoring thresholds may also be displayed on the user interface. While specific examples of first modes of operation, second modes of operation, and transition conditions are provided in display areas 402, 404, and 406, one of skill in the art will appreciate that other modes of operation and other transition conditions are contemplated within the scope of the disclosure.

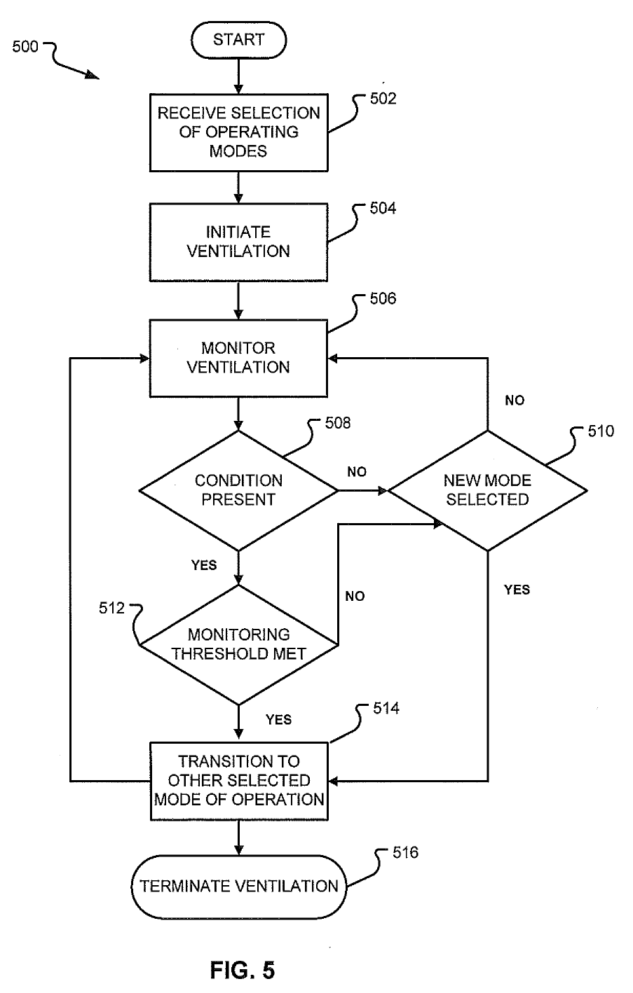

FIG. 5 is a flow chart representing an embodiment of a method 500 for operating a multimode ventilator with user-selected modes of operation. Flow begins at operation 502 where the multimode ventilator receives the user's selections. In embodiments, the multimode ventilator receives selections of one or more operating modes, transition conditions, transition thresholds, and monitoring thresholds according to an embodiment of the method described with respect to FIG. 2. Upon receiving the selections of one or more operating modes, transition conditions, transition thresholds, and monitoring thresholds, flow proceeds to operation 504 where the ventilation is initiated on a patient.

Flow then proceeds to operation 506 where the multimode ventilator monitors the ventilation of the patient. At operation 506, the multimode ventilator may monitor conditions including, but not limited to airflow, FIO₂ percentages, respiration rate, air saturation levels, end-tidal CO₂ or CO levels, spontaneous efforts made by the patient, air flow, volume reading, pressure levels, S_{P}O₂ levels. Other breathing and/or ventilation conditions known to the art may also be monitored at operation 506. In embodiments, monitoring by the ventilator may be continuous. In other embodiments, the monitoring performed at operation 506 may be periodically performed.

The conditions monitored at operation 506 may be recorded in stored in memory such as memory 112 for later review by a physician of clinician. In further embodiments, the monitored conditions and/or their associated readings may be displayed on a user interface for review by a physician or clinician.

The method 500 further includes a decision operation 508 in which the monitored conditions are tested and compared against the transition conditions If the monitoring conditions do not meet the transition conditions, flow branches “NO” to operation 510. Decision operation 510 represents a user override of the current ventilation mode. At decision operation 510, the ventilator checks to determine whether a new mode of operation has been selected by a user. If a new mode of operation has been selected manually by the user, flow branches “YES” to operation 514, the multimode ventilator transitions to the new mode of operation, and then flow returns to operation 506 and the multimode ventilator continues to monitor the patient. If the user does not provide a new mode at decision operation 510, flow branches “NO” to operation 506 and the multimode ventilator continues to monitor the patients.

If, in the first decision operation 508, the monitoring conditions meet the transition conditions, flow branches “YES” to operation 512. Operation 512 represents a confirmation operation in which the ventilator confirms that the transition conditions are consistent enough to cause a transition. At decision operation 512, the ventilator determines whether the monitoring threshold has been met. In embodiments, the ventilator may not transition between modes of operation upon initially meeting a transition condition. In such embodiments, the transition condition must be consistently met before transitioning to a different mode of operation. At decision operation 512, the multimode ventilator determines whether the monitoring threshold has been met. In embodiments in which the transition condition must be present for a certain period of time, decision operation 512 determines whether the period of time has been met. In such embodiments, the multimode ventilator may begin a timer upon first reaching the transition condition at operation 512. Upon subsequently meeting the transition condition, operation 512 checks the timer to ensure that the monitoring threshold has been met. If the monitoring threshold has not been met, then flow branches “NO” to operation 510 and flow continues as described above.

If the monitoring threshold has been met, flow branches “YES” to operation 514, in which the multimode ventilator transitions to another mode of operation, and flow proceeds to operation 506. In embodiments using a timer, if any monitored condition fails to meet the transition condition between the first instance of the monitored condition meeting the transition condition and the completion of the monitoring threshold, the timer may be reset.

Eventually, the multimode monitor receives a signal to terminate ventilation, and ventilation is terminated at operation 516.

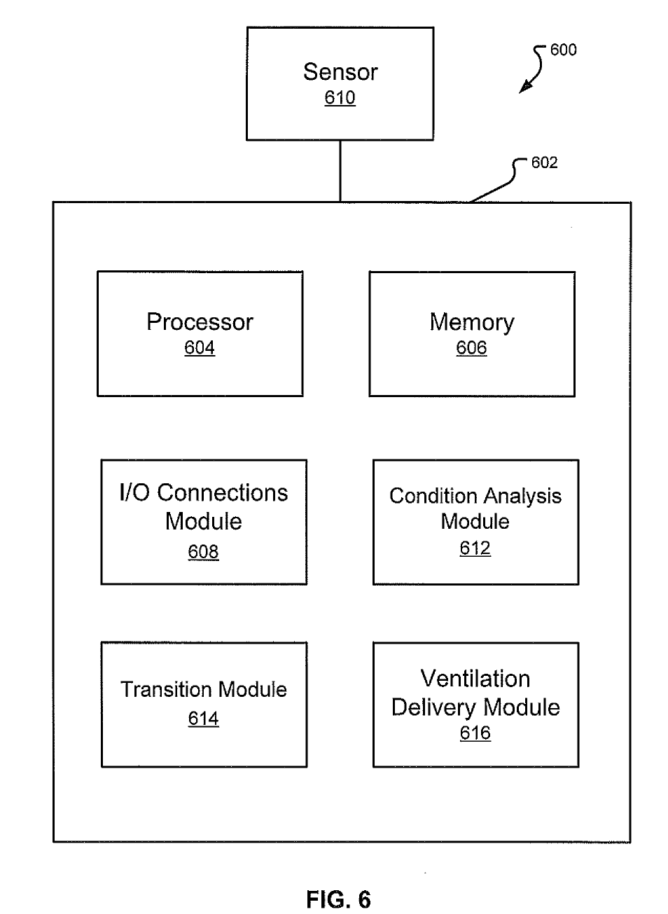

FIG. 6 illustrates a functional block diagram of modules and other components that may be used in an embodiment of a multimode ventilator 600. The ventilator 602 includes various modules 610-616, memory 606 and one or more processors 604. Memory 606 is defined as described above for memory 112. Similarly, the one or more processors 604 are defined as described above for the one or more processors 116.

In embodiments, I/O Connections Module 608 is used to facilitate interaction between the multimode ventilator 602 and input and output devices, such as the input and output devices described with respect to FIG. 1. In embodiments, I/O Connections Module 608 is adapted to display user interfaces, such as the embodiments of user interfaces described with respect to FIGS. 3 and 4, and to receive user input entered using the contemplated user interfaces. In embodiments I/O Connections Module 608 is capable of communicating user input to processor 604, memory 606, and/or the other modules 612-614 of ventilator 602.

Sensor 610 conducts measurements to determine the monitored conditions as described with respect to FIG. 5. In embodiments, sensor 610 can include one or more sensors configured to detect conditions including, but not limited to airflow, FIO₂ percentages, respiration rate, air saturation levels, end-tidal CO₂ or CO levels, spontaneous efforts made by the patient, air flow, volume reading, pressure levels, S_{P}O₂ levels. In other embodiments, any other sensors known to the art may be employed with ventilator 602 to detect other characteristics and conditions.

Sensor 610 communicates the monitored conditions to Condition Analysis Module 612. In embodiments, Condition Analysis Module 612 is capable of testing the monitored conditions against the threshold conditions received from the user via I/O Connection Module 608 or, in other embodiments, automatically generated by ventilator 602. In further embodiments, Condition Analysis Module 612 is also capable of determining whether or not the monitoring threshold has been met. In such embodiments, Condition Analysis Module 612 may maintain a timer, a counter, or any other means known to the art to determine whether or not a monitoring threshold has been met. In other embodiments, Condition Analysis Module 612 is capable of determining whether a new mode of operation has been selected by a user during operation of the ventilator 602.

Upon determining that a threshold condition and monitoring condition has been met, Condition Analysis Module 612 communicates with Transition Module 614. In other embodiments, Condition Analysis Module 612 may communicate with Transition Module 614 when the user changes the mode of operation during the operation of ventilator 602. Transition Module 614 performs the transition between modes of operation. For example, in embodiments, Transition Module 614 may change the operation of ventilator 602 from a control mode to a spontaneous mode upon indication that the required conditions and thresholds have been met from Condition Analysis Module 612. In further embodiments, Transition Module 614 may also be used to initiate ventilation of the patient.

Transition Module 614 communicates operation instructions to Ventilation Delivery Module 316. Ventilation Delivery Module performs the ventilation dictated by the mode of operation. Multimode ventilator 600 is an embodiment of a multimode ventilator contemplated within the scope of the present disclosure. One of skill in the art will appreciate that ventilators incorporating different types and/or amounts of modules may be employed within the scope of the present disclosure.

While various embodiments have been described for purposes of this disclosure various changes and modifications may be made which are well within the scope of the present invention. For example, other modes of operating a ventilator known to the art but not specifically described in the present disclosure can be practiced with the embodiments disclosed herein. Furthermore, the embodiments described herein are scalable such that more than two modes of operation may be selected. In other contemplated embodiments, a ventilator may incorporate the use of interrupts to perform transitions between modes of operation rather than relying on timers and/or counters to determine whether threshold requirements have been met.

In further embodiments, the disclosed user interface may be include warnings and/or notification to inform the user when the multimode ventilator transitions between modes of operation In yet another embodiment, the multimode ventilator tracks statistics related to the operation of the ventilator. Such statistics may include, but are not limited to, a percentage of time that the ventilator has operated in a certain mode of operation, the number of times the ventilator has transitioned between modes of operation, etc. Numerous other changes may be made which will readily suggest themselves to those skilled in the art and which are encompassed in the spirit of the disclosure.

This disclosure described some embodiments with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the operating system or hardware level. In this regard, any number of the features of the different embodiments described herein may be combined into one single embodiment and alternate embodiments having fewer than or more than all of the features herein described are possible. Similarly, one of skill in the art will readily appreciate that functional elements disclosed herein may be performed using software implementations, hardware implementation, or a combination of both.

Although the embodiments have been described in language specific to structural features, methodological acts, and computer-readable media containing such acts, it is to be understood that the possible embodiments, as defined in the appended claims, are not necessarily limited to the specific structure, acts, or media described. One skilled in the art will recognize other embodiments or improvements that are within the scope and spirit of the present disclosure. Therefore, the specific structure, acts, or media are disclosed only as illustrative embodiments.

What is claimed is:

1. A method for transitioning between a first mode of ventilator operation and a second mode of ventilator operation, the method comprising:
providing a plurality of first modes of ventilator operations and a plurality of second modes of ventilator operations;
receiving a selection of the first mode of ventilator operation;
receiving a selection of the second mode of ventilator operation;
initiating ventilation of a patient in the first mode of ventilator operation;
monitoring the ventilation of the patient; and
transitioning into the second mode of ventilator operation.

2. The method of claim 1, further comprising receiving one or more conditions operable to initiate the transition between the first mode of ventilator operation and the second mode of ventilator operation.

3. The method of claim 2, further comprising receiving one or more conditions operable to initiate the transition between the second mode of ventilator operation and the first mode of ventilator operation.

4. The method of claim 2, further comprising collecting monitored data from the patient.

5. The method of claim 4, further comprising comparing the monitored data against the received conditions.

6. The method of claim 5, wherein the step of transitioning into the second mode of ventilator operation is based on the comparing of the monitored data against the received conditions.

7. The method of claim 1, further comprising generating a user interface for displaying the plurality of first and second modes of ventilator operations.

8. The method of claim 7, wherein the user interface displays notification informing the user of a transition between the first mode and the second mode of operations.

9. A method for transitioning between a first mode of ventilator operation and a second mode of ventilator operation, the method comprising:
providing a plurality of first modes of ventilator operations and a plurality of second modes of ventilator operations;
receiving a selection of the first mode of ventilator operation;
receiving a selection of the second mode of ventilator operation;
receiving a first selection of first transition conditions operable to initiate the transition between the first mode of ventilator operation and the second mode of ventilator operation;
initiating ventilation of a patient in the first mode of ventilator operation;
monitoring the ventilation of the patient; and
transitioning into the second mode of ventilator operation based on detection of the first transition conditions.

10. The method of claim 9, wherein the first transition conditions comprises one of:
an FIO₂ percentage;
an end-tidal CO₂ level;
a P100 level;
an S_{P}O₂ level;
a Minute Ventilation requirement; and
a respiratory rate criteria.

11. The method of claim 9, wherein the first transition condition specifies when the ventilator transitions from the first mode of operation to the second mode of operation.

12. The method of claim 9, further comprising receiving a second selection of a second transition condition.

13. The method of claim 12, wherein the second transition condition specifies when the ventilator transitions from the second mode of operation to the first mode of operation.

14. The method of claim 12, wherein the second transition condition comprises one of:
a respiration rate;
an end-tidal CO level;
a minimum threshold of spontaneous effort; and
an airflow rate.

15. The method of claim 9, further comprising providing a plurality of monitoring thresholds.

16. The method of claim 15, further comprising receiving a selection of a first monitoring threshold.

17. The method of claim 16, further comprising receiving an association between the first monitoring threshold and the first transition condition.

18. A computer storage medium encoding computer-readable instructions executable by a processor for performing a method of transitioning between a mandatory mode of ventilator operation and a spontaneous mode of ventilator operation during the operation of a ventilator on a patient, the method comprising:
providing a plurality of mandatory modes of ventilator operation for selection by a user;
proving a plurality of spontaneous modes of ventilator operation for selection by the user;
providing one or more mode transition conditions;
receiving, from the user, a selection of a first mandatory mode of ventilator operation from the plurality of mandatory modes, a selection of a first spontaneous mode of ventilator operation from the plurality of spontaneous modes, and a selection of a first transition condition and a second transition condition;
operating the ventilator in the selected first mandatory mode of ventilator operation;
monitoring the ventilation of the patient;
transitioning operation of the ventilator from the selected first mandatory mode of ventilator operation to the selected first spontaneous ventilator operation if the first transition condition is met.

19. The computer storage medium claim 18, wherein the first transition condition specifies when the ventilator should transition from the first mandatory mode to the first spontaneous mode, and wherein the second transition condition specifies when the ventilator should transition back from the first spontaneous mode to the first mandatory mode.

20. The computer storage medium of claim 18, further comprising receiving, from the user, a selection of a first monitoring threshold,

21. The computer storage medium of claim 20, wherein the first monitoring threshold is associated with the first transition condition.

22. The computer storage medium of claim 21, wherein the first monitoring threshold is a unit of time.

23. The computer storage medium of claim 24, wherein the transitioning of the ventilator from the selected first mandatory mode of ventilator operation to the selected first spontaneous ventilator operation when the first transition condition is met for the duration of the first monitoring threshold.

24. The computer storage medium of claim 21, further comprising receiving, from the user, a selection of a second monitoring threshold, wherein the second monitoring threshold is associated with the second transition condition.

25. The computer storage medium of claim 24, wherein the first monitoring threshold is different than the second monitoring threshold.

26. A multimode ventilator comprising:
a display for displaying:
a plurality of mandatory modes of ventilator operation;
a plurality of spontaneous modes of ventilator operation; and
one or more mode transition conditions;
a user input device for receiving from a user a selection of a first mandatory mode of ventilator operation from the plurality of mandatory modes, a selection of a first spontaneous mode of ventilator operation from the plurality of spontaneous modes, and a selection of a first transition condition;
a ventilation delivery module for performing ventilation on a patient, wherein the ventilation delivery module is adapted to operate in any of the mandatory modes and spontaneous modes of ventilator operation;
an analysis module for analyzing patient respiratory activity against the first transition condition; and
a transition module for identifying the selected first mandatory mode and the selected first spontaneous mode and transitioning operation of the ventilator between the selected first mandatory mode of ventilator operation and the selected first spontaneous mode of ventilator operation based upon information received from the analysis module.

27. The multimode ventilator of claim 26, wherein the first transition condition is associated with the first mandatory mode of ventilator operation.

28. The multimode ventilator of claim 26, wherein the user input device further receives a selection of a first monitoring threshold.

29. The multimode ventilator of claim 28, wherein the analysis module further determines if the patient respiratory activity meets the first transition condition.

30. The multimode ventilator of claim 29, wherein if the analysis module determines that the patient respiratory activity meets the first transition condition, the analysis module further determines whether the first monitoring threshold has been met.
